(19) United States
(12) Patent Application Publication
(10) Pub. No.: US 2020/0297936 A1
(43) Pub. Date: Sep. 24, 2020

(54) DISPOSITIF D'INJECTION MANUELLE

(71) Applicant: EDIX SA, Luxembourg (LU)

(72) Inventors: Roland CHERIF-CHEIKH, Castelldefels-Barcelona (ES); Tabatha BOURGOIS, Barcelona (ES); Lluis PARETA BELTRAN, Castellbisball-Barcelona (ES); Frederic LACOMBE, Sant Cugat del Valles (ES); Laurence LACHAMP, Gava Barcelona (ES)

(21) Appl. No.: 16/755,908

(22) PCT Filed: Oct. 16, 2018
(86) PCT No.: PCT/EP2018/078287

§ 371 Date: May 22, 2020

(30) Foreign Application Priority Data
Oct. 17, 2017 (FR) 17/59714

(51) Int. Cl. A61M 5/315; A61M 5/32

(52) U.S. Cl. CPC A61M 5/31535; A61M 5/31528; A61M 2005/3114; A61M 5/31551; A61M 5/3204

(57) ABSTRACT

Disclosed is a device for filling the injection device, the injection device of the type including a cylindrical body enclosing a reservoir containing a product to be injected, the reservoir opening at one end on an injection needle and being closed at its other end by a plunger mounted so as to be movable in translation in the reservoir under the control of a plunger rod provided with a control member, the device including a unit for controlling the quantity of product to be delivered. The control unit is arranged on the body in an area located outside an area where the body is intended to be fully gripped by the hand. Also disclosed is a device for filling the injection device.

### BACKGROUND OF THE INVENTION

### Field of the Invention

The present invention relates to improvements to manual injection devices with a view to improving especially both their ease and precision of use.

Manual injection devices shall be understood hereinafter to be injection devices, such as syringes or equivalent tools, intended for the administration of medical treatments such as those delivered intradermally, subcutaneously, intramuscularly, intravenously, topically, transcutaneously or by any other known route of administration.

### Description of the Related Art

It is known that many treatments require the administration of products by injection. These are mainly therapeutic or prophylactic treatments, but also include other applications such as certain aesthetic, cosmetic, dermatological, dental or orthopaedic treatments. Apart from use in humans, injections are also increasingly finding applications within the scope of veterinary treatments.

In general, the medical sector is facing a trend towards a growing need for injectable treatments. Similarly, in the near future, it is expected that the demand for innovative solutions for performing these injections will increase in order to improve user comfort and to respond to the new problems posed by the injection of products that are increasingly complex and less easy to administer, due especially to their high viscosity, high concentrations, or stability problems before or during injection.

Moreover, it is known that more and more treatments are on-going and many are, or could be, advantageously administered outside the hospital or even at the patient's home. It is therefore important to improve the patient's injection experience in order to ensure compliance with treatment, especially since it is known to the medical profession that a significant proportion of patients do not fully comply with their prescriptions for reasons ranging from fear of needles, injection pain, or lack of a healthy injection site to inject their treatment daily.

Various studies have been carried out to analyse patient expectations in order to identify possible improvements in the field of injections, as well as the specific areas that could benefit as a result of these improvements.

These studies have shown that the basic needs can be summarised as follows:
manual injection with greater force and better control of this injection force and speed, even for highly viscous or pasty products, and thus the ability to use finer needles than those required for usual manual injections of the type used on syringes or other injection devices,
improvement in the accuracy and control of manual injection devices especially for injection volumes and speeds that are lower or more difficult to control with current manual injection devices, such as syringe-type devices,
better definition of the injection sites and the stability of the inserted needle even with longer insertion times and/or with different injection mechanisms than with manual injections of the syringe type,
possibility to achieve this with conventional containers and the usual manufacturing processes used for manual injection devices, for example of the syringe type.

In order to meet these fundamental needs, multiple devices have been proposed that seek to improve the handling of injection devices, reduce the size of needles, control the speed of administration or control the injection forces, some of which are alternatives to syringes, such as injection pumps, auto-injectors, pen-injectors etc.

It is also known that to perform or control injection from a reservoir closed by a movable plunger, means using rotational injection mechanisms have been widely tried and tested, as described in U.S. Pat. No. 2,283,915.

In addition, a rotational injection mechanism has been proposed in U.S. Pat. No. 4,189,065, using a threaded plunger rod of which the rotational drive in a nut integral with a syringe causes the plunger to move longitudinally in the syringe.

It should be noted that for medical and nursing specialists, an injection is done by translation, whereas for the general population, this gesture is much less common, natural and simple than rotation. Indeed, injection in translation is an atypical gesture that is never performed in everyday life. This gesture requires a precise positioning of three fingers. This translation can give the patient the impression of something being introduced into his/her body by the movement of the thumb under pressure towards the skin. This gesture performed without a fixed support poses a problem of stability which leads to movements of the inserted needle and hinders the precision of the dosages. This gesture is sensitive to the pressure received in return, which can be perceived as variable, making it difficult to perform at a constant speed from start to finish. Finally, in this unstable holding position, pauses during the injection depending on the pain felt are difficult to achieve.

Rotational injection is a habitual gesture carried out in everyday life, which can be performed without precise positioning and with different fingers or even with the hand. This rotation avoids the impression of introducing something into the body and drastically reduces the pressure towards the skin. This gesture is performed with a fixed support that stabilises the device and the needle, is not sensitive to pressure, and is naturally performed at a constant speed. In this stable holding position, pauses depending on perception are easy to achieve.

Another constraint related to injections, especially when performed by non-specialists and involving viscous or pasty products, is knowing when the entire dose has been injected. Faced with this uncertainty, the tendency is to press very hard at the end of the injection, causing pain through pressure and needle movements in the tissues.

With injections in translation, the visibility of the plunger at the bottom of the syringe reservoir very close to the injection point is also difficult, whereas with rotational injections, such as those carried out with a device according to the invention, it is impossible to force under pressure and it is possible to have a precise and visible stop point of the rotation which signals the end of the injection. In addition, the graduations are located higher up on the body of the device, where they are much easier to read than at the base, in contact with the skin.

In the case of pressure auto-injectors that work in translation, these devices exert a strong pressure on the skin, which is difficult for patients to accept and tolerate, especially for children and the elderly, for whom this represents a veritable jab-like or shock-like sensation.

### SUMMARY OF THE INVENTION

The aim of the present invention is to propose new injection devices which retain the useful properties of auto-injectors and pen-injectors, but which remain simple, small, lightweight and robust and therefore easily transportable, like conventional injection devices of the syringe type.

The new devices according to the invention may also be used to perform administrations other than injections, for example topical, mesotherapy-like, or intramammary (in veterinary medicine) administrations.

The present invention therefore relates to an injection device of the type comprising a cylindrical body enclosing a reservoir containing a product to be injected, said reservoir opening at one end on an injection needle and being closed at its other end by a plunger mounted so as to be movable in translation in the reservoir under the control of a plunger rod provided with a control member, the device comprising means for controlling the quantity of product to be delivered, characterised in that the control means are arranged on the body in an area located outside an area where the body is intended to be fully gripped by the hand, this area intended to be fully gripped by the hand preferably being located in the distal area the body.

The plunger rod may comprise a threaded portion on which an adjusting wheel is screwed, and the body may comprise two end stops, namely a distal stop and a proximal stop, between which the adjusting wheel can be positioned.

This adjusting wheel may comprise graduations on its periphery which are visible at the proximal end of the body, and the body may comprise at least one index whose cooperation with the graduations makes it possible to determine an angle of rotation of said adjusting wheel and thus its longitudinal displacement in relation to the threaded portion. The area of the adjusting wheel comprising the graduations may protrude laterally from the body in at least one area.

In addition, the proximal part of the body may cover at least part of the adjusting wheel and may comprise at least one transparent area through which at least one of the graduations can be seen.

The proximal part of the body may form a cylindrical housing, especially open on one of its sides, which is traversed by a threaded portion of the plunger rod, the adjusting wheel being able to move by screwing or unscrewing on the threaded portion so as to pass from the proximal stop to the distal stop, these stops being constituted, for example, by the two end walls of the housing.

Lastly, the edge of the adjusting wheel may constitute an index, the cooperation of which with graduations on the body will make it possible to determine an angle of rotation of said adjusting wheel and thus its longitudinal displacement in relation to the threaded portion.

The reservoir of the device according to the invention may be constituted by a standard, single or double-chamber cartridge or injection syringe, the plunger rod of which may comprise an anti-rotation element such as a longitudinal groove, a flattening on its threaded portion in contact with a complementary part of the body or housing, or an element that acts frictionally on the internal wall of said reservoir, such as an O-ring especially.

In one embodiment, the distal part of the body of the injection device may comprise an extension element, which may end in a tapered part, which will be traversed by the cannula of the injection needle, this cannula extending beyond the distal part of the extension element by a length equal to the desired insertable length of said cannula.

According to the invention, the plunger rod of the injection device may comprise at least one threaded portion which will be engaged with a nut integral with the body, and the control member will be in the form of a bell extending towards the distal end of the body and capable of covering part of the latter.

The present invention also relates to an injection needle comprising a cannula fixed to a base, this base possibly comprising an axially centred circular plate with an adjacent cylindrical boss of the same axis, hollowed out by a cavity in communication with the channel of the needle, this cavity forming a convergent duct going from its proximal part towards its distal part.

In a variant, the injection needle comprises a cannula fixed to a cylindrical base formed by a tubular element comprising a duct which is arranged in the extension of the duct of the cannula, and the internal diameter of which is greater than that of the cannula, the external diameter of the base possibly being greater than that of the cannula.

In one embodiment, the circular plate of the needle base is applied against the distal face of a single or double-chamber reservoir, especially cartridge, by holding means, especially crimping means. In an especially beneficial way, the connection between this base and the distal face of the reservoir is established in a non-tight manner, so that, when the needle is inserted into a vein, the blood pressure allows air to escape and blood to rise in the distal part of this reservoir.

The injection device may comprise a cylindrical body enclosing a reservoir, especially constituted of a single or double-chamber cartridge or a syringe, containing a product to be injected, which is open at one end on an injection needle and is closed at its other end by a plunger mounted so as to be movable in translation in the reservoir under the control of a plunger rod provided with a control member, the body comprising means, possibly removable, for holding the reservoir, and the length of the body being such that it allows passage of a cannula length equal to the length that it is to be inserted. The plunger rod may comprise at least one threaded portion which is engaged with a nut integral with the body, the control member being bell-shaped extending towards the distal end of the body and being capable of covering part of the body.

The present invention also relates to a device for filling a single or double-chamber reservoir, especially cartridge or syringe, with a product to be injected, having a storage container for the product to be injected comprising a filling nozzle suitable for insertion inside the neck of said reservoir. Sealing means are provided between said neck and said nozzle, these sealing means possibly consisting of an O-ring arranged in a circular groove between the neck of the reservoir and the nozzle.

The present invention also relates to a method for filling a single or double-chamber reservoir, especially a cartridge or a syringe, equipped with a plunger and terminating at its distal end in a neck, with a treatment product, the method comprising the steps consisting of:
positioning the plunger at the distal end of the reservoir,
introducing a nozzle of a storage container containing the treatment product to be injected into the neck of the reservoir,
transferring the product to be injected from the storage container into the reservoir, the plunger thereof being pushed by said product to be injected.

The present invention also relates to a single or double-chamber reservoir, especially cartridge or syringe, comprising a product to be injected, a reservoir neck and a plunger, in which the product to be injected extends into the reservoir between its neck and the plunger without any air space between said neck and said plunger.

The present invention also has relates to a device for filling a product to be injected into a single or double-chamber reservoir, especially cartridge or syringe, comprising a plunger and at its distal end an injection needle, having a receiving housing of cylindrical shape which is pierced at each of its ends by a longitudinal cylindrical receptacle, namely a receptacle formed in its proximal part which is capable of receiving the reservoir, and a receptacle formed in its distal part which is capable of receiving a filling container closed by a septum and comprising means for propelling the treatment product, stop means being provided for immobilising the reservoir in the housing in a position such that its needle has perforated the septum and penetrated the filling container. Preferably the receiving housing is equipped with means capable of allowing the position of the plunger of the reservoir to be seen when it is in place in its receptacle, and with means capable of measuring a displacement of the plunger.

The injection device according to the invention may comprise a plunger driven by a plunger rod under the action of a control head and, at its distal end, an injection needle, and the proximal end of the plunger rod may comprise means capable of cooperating successively, in a removable manner, with, on the one hand, a pressure plunger control head and, on the other hand, a rotary plunger control head. Preferably the plunger rod is of circular cross-section, and the injection device comprises a nut capable of being fixed to the proximal part of the reservoir, the diameter of the threaded hole of which nut is greater than that of the plunger rod and smaller than the internal diameter of the reservoir.

In such an injection device the pressure plunger control head consists of a cylindrical element of which the diameter is smaller than that of the threaded hole of the nut, so that it can be received in the reservoir, this cylindrical element being capable of being fixed to the proximal end of the plunger rod.

In the same injection device the rotating plunger control head comprises a screw capable of screwing into the threaded hole of the nut, so as to come to rest on the proximal part of the plunger rod and be able to push it towards the distal end of the reservoir when the screw is screwed in. This screw is preferably integral with the bottom of a cap.

The present invention also relates to a device for filling and packaging a set of single or double-chamber reservoirs, especially cartridges or syringes, comprising a receiving tub provided with means capable of holding a number of rows of reservoirs in a vertical position, and a cover capable of closing the latter, comprising:
aligning means capable of keeping the cover in the same lateral position with respect to the reservoirs,
resilient means capable of pushing the cover back to an open position,
means provided on the inner face of the cover of the receiving tub for holding above each reservoir a plunger suitable for being received in that container.

The present invention also relates to a method for filling and packaging a set of single or double-chamber reservoirs, especially cartridges or syringes, with a product in a device comprising a receiving tub provided with means capable of holding a number of rows of reservoirs in a vertical position, and a cover capable of closing the latter and comprising on its lower face plungers held in line with each reservoir, the method comprising the steps consisting of:
filling the reservoirs contained in the receiving tub with the product,
carrying out a possible treatment of the product,
forming a vacuum in the receiving tub,
closing the lid so that the plungers are pushed into the reservoirs,
breaking the vacuum to keep the plungers in the reservoirs,
opening the lid.

### BRIEF DESCRIPTION OF THE DRAWINGS

Some embodiments of the present invention will be described hereinafter by way of non-limiting example, with reference to the accompanying drawings, in which:

FIG. 1 is a front view in axial and longitudinal section of a first embodiment of an injection device according to the invention,

FIG. 1a is an enlarged view of the distal end of the injection device shown in FIG. 1,

FIG. 2 is a top view of the injection device shown in FIG. 1,

FIG. 3 is a sectional view of the injection device shown in FIG. 1 along the line AA thereof,

FIG. 4 is a left-hand perspective view of the injection device shown in FIG. 1,

FIG. 5 is a perspective view of a full gripping by hand of the injection device shown in FIGS. 1 to 4,

FIG. 6 is a front view in axial and longitudinal section of a variant of the injection device shown in FIGS. 1 to 5,

FIG. 6a is a schematic view of the indexing means implemented on the injection device shown in FIGS. 6 and 7,

FIG. 7 is a perspective view of the injection device shown in FIG. 6,

FIG. 8 is an axial and longitudinal sectional view of a second embodiment of an injection device according to the invention,

FIG. 8a is a longitudinal sectional view on an enlarged scale of an embodiment of an injection needle according to the invention,

FIG. 8b is an enlarged partial axial and longitudinal sectional view of the needle shown in FIG. 8a,

FIG. 9 is a top view of the injection device shown in FIG. 8,

FIG. 9a is a perspective view of a full gripping by hand of the injection device shown in FIGS. 8 and 9,

FIG. 10 is an axial and longitudinal sectional view of a variant of an injection device according to the invention,

FIG. 10a is an axial and longitudinal sectional view on an enlarged scale of a needle suitable for use in the injection device shown in FIG. 10,

FIGS. 11 and 12 are axial and longitudinal sectional views of an injection device with rotational injection control before and after injection respectively,

FIG. 11a is a partial axial and longitudinal sectional view of the distal part of a variant of the injection device shown in FIG. 11,

FIG. 13 is a partial axial sectional view of a device for filling a single or double-chamber cartridge or syringe,

FIGS. 14 and 15 are views of another device for filling a single or double-chamber cartridge or syringe, in axial and longitudinal section and plan view respectively,

FIGS. 16a to 16c are partial views of the proximal end of a syringe capable of performing two possible injection modes, namely by pressure and by rotation,

FIG. 17 is a cross-sectional view of a device for packaging a series of single or double-chamber cartridges or syringes,

FIG. 18 is a perspective view of an embodiment of the invention,

FIG. 19 is an axial and longitudinal sectional view of the embodiment shown in FIG. 18,

FIG. 20 is an axial and longitudinal sectional view of an embodiment of an injection device according to the invention shown in the rest or pre-injection position,

FIG. 20a is an enlarged view of area A of the injection device as shown in FIG. 20,

FIG. 21 is a perspective view of the injection device shown in FIG. 20,

FIG. 22 is an axial and longitudinal sectional view of the injection device of FIG. 20 shown during injection,

FIG. 22a is an enlarged view of area B of the injection device as shown in FIG. 22,

FIG. 23 is an axial and longitudinal sectional view of the injection device of FIGS. 20 and 22 shown after the injection has been completed,

FIG. 23a is an enlarged view of area C of the injection device as shown in FIG. 23.

### DESCRIPTION OF THE PREFERRED EMBODIMENTS

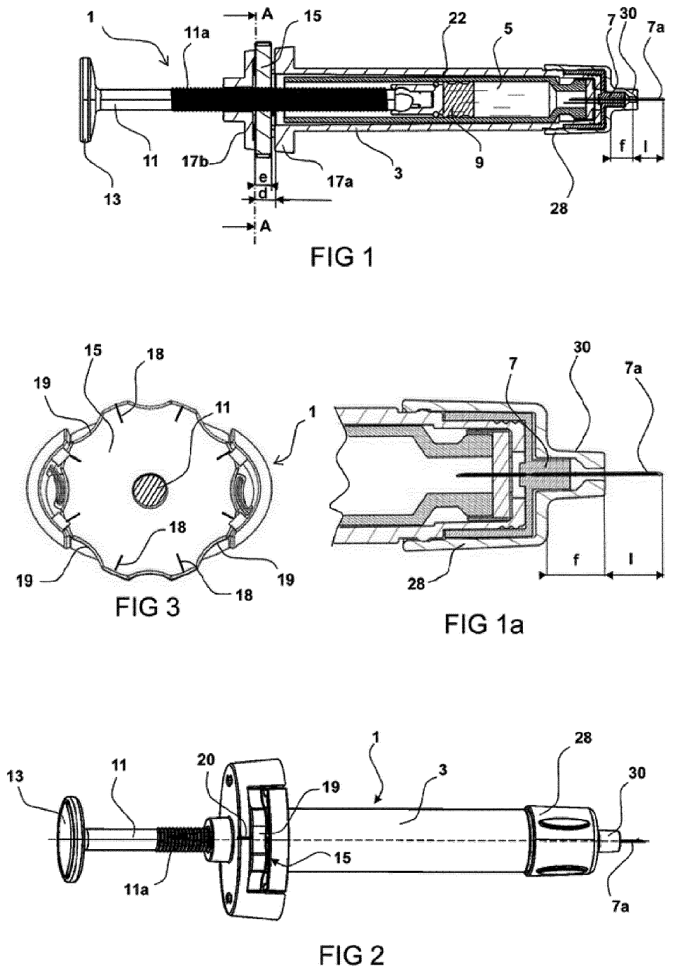

FIGS. 1 to 5 show a first embodiment of an injection device according to the invention, which is primarily intended for carrying out intradermal or subcutaneous injections.

This injection device 1, which is embodied similarly to a syringe, has a tubular body 3, inside which is arranged a reservoir 5 containing a product to be injected and which ends in an injection needle 7 provided with a cannula 7a. In a known manner, a plunger 9 is slidingly mounted inside the reservoir 5 under the action of a plunger rod 11, the outer end of which, referred to as the proximal end in the present text, comprises an injection control head 13.

In order to ensure good contact between plunger 9 and reservoir 5 and to obtain good dosing precision, as well as to resist the pressures involved, for example when injecting viscous or pasty products, it is advantageous to use, in accordance with the invention, a solid plunger of the conventional type as used for example in dental cartridges or in certain pen-injectors.

The piston rod 11 comprises a threaded portion 11a on which a nut is screwed which forms an adjusting wheel 15, of thickness e, which is longitudinally displaceable between two stops of the tubular body 3, namely a distal stop 17a and a proximal stop 17b, which are spaced apart by a distance d which is dependent on the maximum quantity of product which it is desired to be able to inject, as described hereinafter.

In a specific embodiment of the invention, the plunger rod 11 is provided on its periphery with a friction element constituted by an O-ring 22 arranged in a groove of the plunger rod, which friction element is applied against the walls of the reservoir 5 so as to create a frictional force preventing the rotation of the plunger rod 11 during the injection or when the device is not in use or when the adjusting wheel 15 is actuated.

This O-ring 22 is able to provide this frictional force without creating a tight barrier, for example by providing openings, not shown in the drawing, in said groove. In this way, the plunger rod 11 can be inserted into the reservoir until it contacts the plunger 9 without trapping air between its distal end and the plunger.

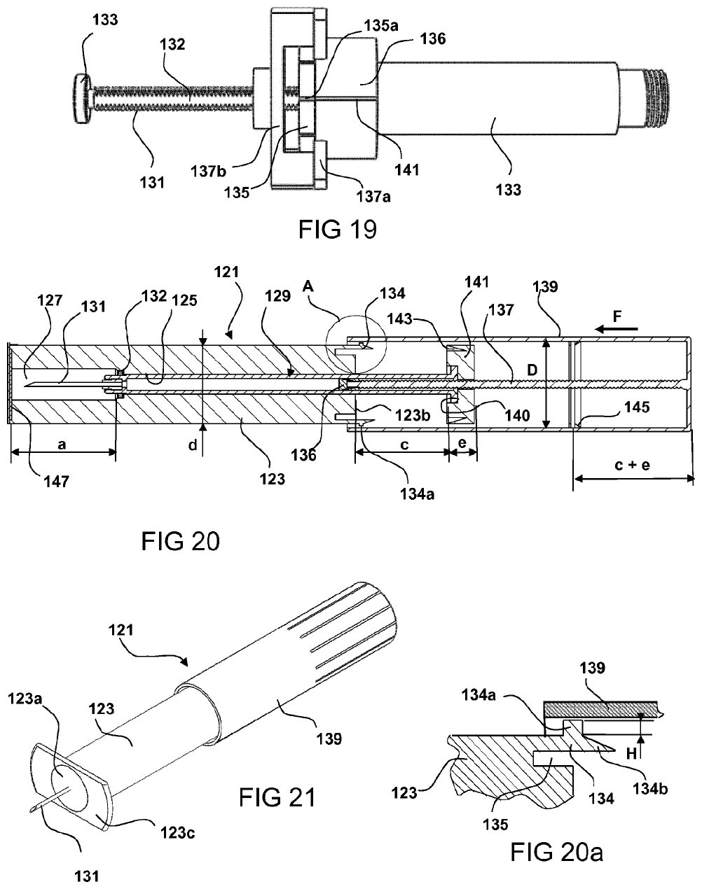

In another embodiment of the invention, which is shown in FIGS. 18 and 19, the plunger rod 11 comprises a longitudinal flattening 12 of which the function is to prevent rotation of the plunger rod during its longitudinal displacement during the injection. For this purpose, at least one of the two stops 17a, 17b comprises a planar portion 12a against which the flattening 12 of the plunger rod 11 is in contact. In this way, the plunger rod 11 cannot rotate and can only move in translation during the injection.

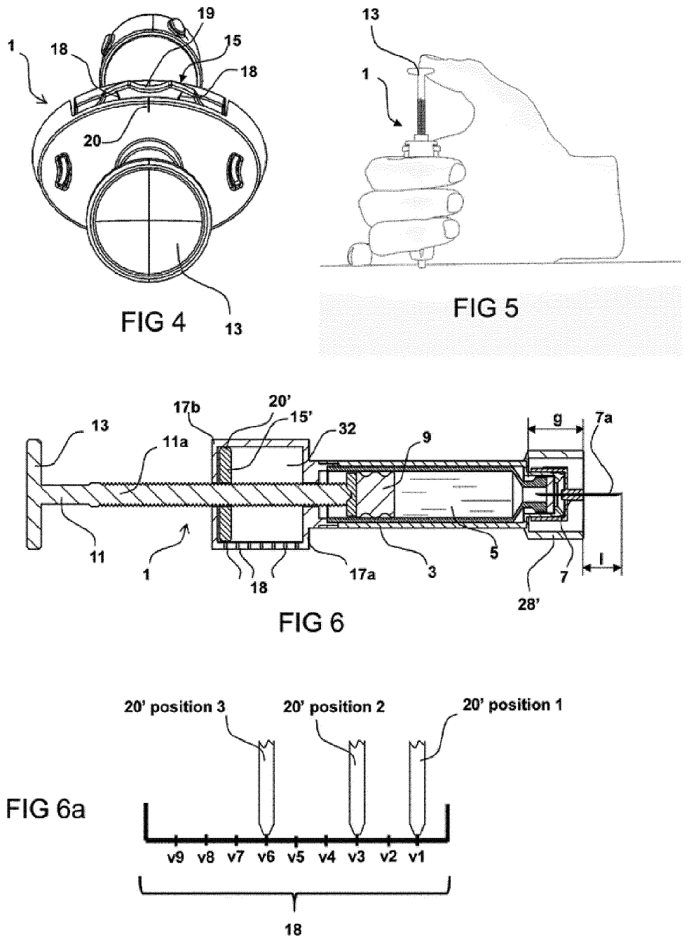

As shown in FIGS. 3 and 4, the adjusting wheel 15 in this exemplary embodiment is in the form of a disc comprising indentations 19 on its periphery intended to improve the user's grip on the wheel 15. The proximal face of the adjusting wheel 15 has on its periphery graduations 18 which are distributed, especially evenly, over its periphery and which are intended to coincide with an index formed on the proximal stop 17b of the body 3. In a variant of this exemplary embodiment, the proximal stop 17b could also be made of a transparent material so that the user can read the graduations 18 through this material.

Under these conditions, after the user has performed the “priming”, i.e. the operation prior to the injection by which he/she evacuates the air contained in reservoir 5 of the injection device, the wheel 15 is in contact with the distal stop 17a. The user then turns this wheel to position it on the selected graduation, which has the effect of moving it back by the distance necessary to eject the desired quantity of product as shown in FIG. 1.

By way of example, if the pitch of the threaded portion 11a is equal to p, and if the wheel 15 comprises N graduations and the diameter of the reservoir 5 is D mm, it is understood that the volume injected for one rotation of the wheel of n divisions will be equal to:

(p×n)/N×π·D2/4

Thus, for a thread pitch p=0.2 mm, a number of graduations N=20 and a reservoir diameter D=8 mm, each graduation will correspond to an injected volume of 0.5 mm3.

The injection device according to the invention can be equipped, as shown in FIGS. 18 and 19, with a reset ring 16. This ring allows the user, if desired, to select a different dose of product to be injected for each administration. For this purpose, the reset ring 16 is rotatably mounted on the body 3 and has an index marker 21 which serves as a starting point for dose selection.

Indeed, once a first dose is selected and administered, the user requires a reference point to perform a reset in order to select a second dose, third dose, etc. Since each dose can vary, the zero of the adjusting wheel 15, which is represented by an index 15a, is found at a different position after each administration. According to the invention, in order to set a new dose, the user will have to position the index 21 of the reset ring 16 opposite the index 15a of the adjusting wheel, then rotate the latter by an angle in relation to the index 21 corresponding to the new desired dose.

The present invention is first of all especially beneficial in that it allows the user to select in advance the quantity of product to be injected into a patient, so that he/she does not have to worry, during the injection, about controlling the quantity of product delivered.

In addition, it has been found that it is possible to inject extremely small quantities of product especially accurately that could not be injected with prior art devices. It is also noted that the injection of these micro-volumes, for example less than 0.05 mL, is not painful for the patient, which for example makes it possible to repeat the injections.

These micro-volume doses (of a few microlitres) are especially useful in many therapeutic areas, such as paediatrics, veterinary (small animals), dermatology (intradermal injection of products against acne, warts and other skin diseases), vaccines or certain botulinum toxin applications, or in cosmetics, for “mesotherapy” type applications. In the case of cosmetic applications, the device may be equipped with one or more very fine needles, for example 0.1 mm in diameter and 0.2 mm in length, or even might not be equipped with a needle but with a dispensing tip. Furthermore, the present invention makes it possible to use the present injection device no longer as a syringe of the conventional type which is held between two fingers and the user's thumb, that is to say in a latero-lateral interdigital grip, but, for example, in a digito-palmar grip, that is to say with the whole hand on the body 3, the user's thumb being positioned on the head 13 of the plunger rod 11 as shown in FIG. 5.

Such a gripping by the hand especially allows easy use for self-administration of controlled volumes of product. This type of gripping by hand also makes it easy to envisage a multi-dose design offering the possibility of injecting these controlled volumes repeatedly, for example for use in mesotherapy or for the application of filler-type products, with variation of the injection sites.

The applicant has also found that this gripping by hand has made it possible to easily and comfortably exert much greater injection forces on the plunger 9 than those that could be exerted with conventional injection devices, and with great dosing precision, moreover without the user having to undergo the usual parasitic movements of conventional injections. This kind of gripping by hand also makes it possible, as a result, to use needles with a much finer cannula for the same product to be injected than those used previously.

Indeed, the present invention makes it possible to separate two functions which have hitherto been confused, namely the gripping function on the one hand and the injection function on the other hand. Thus, according to the invention, the gripping of the injection device is fixed for the entire duration of the injection since it is carried out “by the whole hand”, i.e. by the four fingers of the user's hand except the thumb, and the injection is carried out by the user's thumb. In conventional injection devices, on the contrary, the gripping, which is ensured by the index and middle fingers assisted by the thumb, varies throughout the injection since the thumb, which moves, constitutes one of the elements holding the injection device in place.

However, the applicant has found that this separation of functions makes it easier to exert greater injection forces, which allows the injection of more viscous products such as biopolymeric fillers, such as hyaluronic acid, collagen or elastin, or even polylactic acid (PLA), or to reduce the size of the needle used to inject a given product and, on the other hand, not to exert excessive pressure on the surface of the patient's skin during the injection.

From the above it is understood that this gripping by hand is made possible by the fact that the control of the quantity of the product to be injected is carried out outside the area of the injection device intended to be fully gripped by the hand, i.e. in this example in the distal area of the injection device.

The effectiveness of the whole-handed gripping permitted by the present invention is further improved, in a specific embodiment of the invention, by providing the distal part of the injection device with an extension element 28 making it possible to adjust the injection depth, as shown in FIGS. 1, 1a and 2, this being suitable for attachment, for example by screwing or clipping, to the distal end of the body 3.

This extension element 28 ends forwardly in a tapered part 30 which is traversed by the cannula 7a of a needle 7 and is of the existing known commercial type for pen-injectors. This extension element 28 is of a reduced length and diameter, this diameter being much smaller than the diameter of the body 3, so as to allow the user on the one hand a precise aiming of the injection point and on the other hand to enable him/her to control the desired depth of insertion of the needle cannula. Thus, during the injection, the user will bring the end of the tapered part into contact with the patient's skin, so that the insertion length of the cannula 7a of the needle 7 is equal to the distance 1 by which it protrudes from said tapered part 30.

To adjust the insertion length, it is possible to change either the length f of the tapered part 30 or the length 1 of the cannula of the needle 7.

Thus, according to the invention, the user could have a series of extension elements 28, the lengths f of the tapered part 30 of which will be different and which will enable him/her to adjust the insertable length 1 of the cannula 7a depending on the desired injection depth.

For example, in the case of cosmetic applications, it is possible to adjust the length of the extension element so that the inserted length is so small that the needle cannula itself cannot penetrate the dermis, and the surface of the skin, or stratum corneum, is only penetrated by the tip of the bevel with a thickness of for example 0.10 mm. In this very especial case, the penetration of the cosmetic product into the skin will be improved thanks to these microlesions created by the tip of the cannula of the needle, as for example during mesotherapy treatments, however the product will not be injected, but rather deposited on the surface of the microlesions.

In another embodiment of the invention, the needle can be fixed directly in the extension element 28 so as to form a single piece which will allow the user to have various extension elements with different cannula lengths 1 which he/she will choose depending on the depth of injection he/she wishes to achieve.

It should be noted that this extension element 28 thus performs three functions, namely a function of adjusting the inserted length of the cannula of the needle in the patient's body, a function of stabilising the device during the injection operation since the distal base of its tapered part 30 remains in contact with the patient's body, and a function of fine aiming of the injection point which allows the needle tip to be precisely positioned at the desired location. Indeed, because the device is intended for use with extremely fine needles (29G or more), it is difficult to aim at the injection site because the exposed needle is too fine and therefore not very visible.

The injection device according to the invention thus makes it possible to carry out an intradermal injection with precision, for example in a wrinkle at a determined and reproducible maximum depth. Said length can also be adapted to other precise objectives or injection sites, such as for vaccines or for the intradermal injection of peptides and proteins, products for treating diabetes, such as insulin or GLP-1 analogues, more especially pre-prandial insulin, which has a greater speed of action when injected intradermally.

This injection device according to the invention is especially suitable in the context of paediatric administrations, where the doses of therapeutic products are especially low, leading to reduced volumes. It will be possible, for example, to use a commercial product, and, without changing the concentration, simply, thanks to a device according to the invention, to administer a small dose of the same product with great precision. This will make it possible to keep the same product, which has already been registered, and, without going through all the steps of reformulation and certain phases of clinical studies, which are long and costly, to change the therapeutic area simply thanks to this new specific device.

In a especial embodiment of the invention, a tapered part 30 which completely covers the needle 7, and which can be provided with different shapes and dimensions, is used to constitute a device for topical administration of a product especially useful in the field of ophthalmology, since it allows better stability during administration, without touching the surface of the eye.

In a variant of the invention and as shown in FIG. 1, the reservoir may consist of a cartridge of a standard type, such as dental cartridges or double-chamber cartridges for extemporaneous reconstitution, with a standard volume which may vary between 1 ml and 3 ml and whose manufacture, filling and closure by a septum-type closure element sealed by a ring, has the advantage of being able to be implemented on existing production lines at a reasonable cost.

The present invention makes it possible to provide an injection device which is of a multi-dose type, that is to say that it can deliver, from a reservoir or cartridge, even a double-chamber one, a number of injections of an adjustable volume by successive positioning of the adjusting wheel 15, without having to undergo, between each treatment, a refill of product. This multi-dose device is especially suitable for injecting very small volumes under precise conditions, as in the case of intradermal injection. In addition, it has been found that, due to the small size of the needles and the volume injected, for example less than 0.05 mL, it is possible to perform painless injections, thus allowing multiple injection sites in the same administration area. This type of device according to the invention is for example especially suitable for the administration of products against hyperhidrosis in the palms of the hands or in the soles of the feet, such as botulinum toxin, where it is necessary to repeat many times the injection of small doses of products in the dermis of the patient. It can also be used to inject, intradermally into the skin of the skull of patients suffering from alopecia, therapeutic products that will have an improved efficacy compared to a conventional injection if the intradermal areas in which this type of toxin is effective are targeted.

Such an injection device lends itself especially well to the provision of a disposable device, especially when its reservoir is constituted of a single or double-chamber cartridge, of a standard type, which is then produced in sealed form in the body 3.

The injection device according to the invention can be pre-loaded with the product to be injected. It can also be empty, with the plunger 9 for example in contact with the cartridge neck, and can be loaded at the time of use, for example by means of a filling device described below for filling syringes which correspond to another exemplary embodiment of the invention. Thus, it could be used with a botulinum toxin reconstituted beforehand from its initial form. The contents of this container will then be transferred into the device, for example by means of a syringe.

Such a device may also use a double-chamber cartridge containing a freeze-dried product and its reconstitution medium, with the intermediate plunger being brought to a bypass area for reconstitution directly in the device according to the invention. In this embodiment, the plunger rod 11 will comprise a smooth distal portion to perform the reconstitution, followed by a proximal screw part to perform the administration, as previously described. In order to avoid possible rotation, this plunger rod may also include a flattening to guide the reconstitution.

It is then possible to provide a locking mechanism, for example by means of a key system, for the smooth part of the rod, corresponding to the position of the first plunger (distally) at the location on the lateral bypass area. The user must then remove this locking system to complete the translation of the second plunger (proximally) until contact with the first in order to be able to proceed with the administration of the reconstituted product. Having to remove the locking system forces the user to stop the stroke of the plunger and therefore allows time for proper rehydration once the two products have come into contact. Another variant of the locking mechanism can also be constituted by stops to be passed through when the screw is equipped with flattenings: a first stop to start the plunger travel, and a second stop to make the distal plunger pass through the bypass area before proceeding with the administration of the reconstituted product as described above.

Finally, this device can comprise a system preventing the selection of a dose greater than the one remaining in the reservoir, the screw head can be clipped under the housing above and in contact with the crown when the cartridge is empty. The crown cannot then be turned to select a dose, and the device is locked when empty. It is also possible to provide a precise stop to the rotation of the crown in the form of a pin or stop piece placed on the housing, which will prevent the adjusting wheel 15 from rotating beyond a certain length of plunger corresponding to the maximum dose of product contained in the reservoir that can be administered. Said pin can be moved to the end of a groove on the upper face of the crown, which then fixes this maximum. It is then released only when the crown comes down again once the selected dose has been administered.

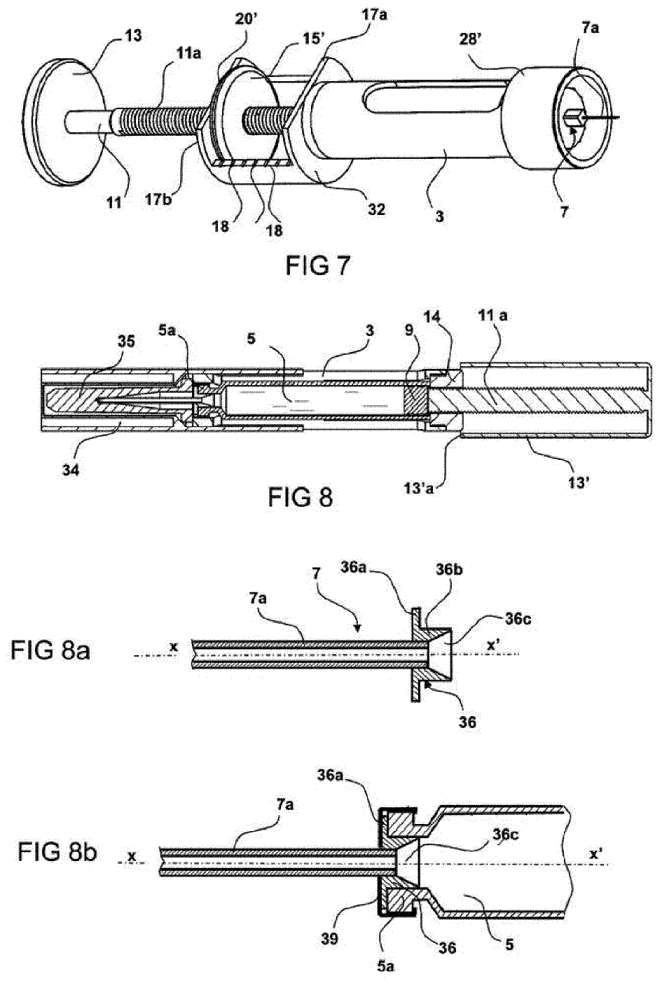

FIGS. 6 and 7 show a variant of the present embodiment of the invention which is especially suitable either for the injection of larger doses of product or for the implementation of multiple injections.

In this device the proximal part of the body 3 forms a cylindrical housing 32, for example open on one of its sides, which is traversed by the threaded portion 11a of a plunger rod 11. In this variant, the adjusting wheel 15′ has the form of a disc and can be moved, as in the previous example in FIGS. 1 to 5, by screwing or unscrewing on the threaded portion 11a to pass from a proximal stop 17b to a distal stop 17a formed, respectively, by the two end walls of the housing 32.

The index here is constituted by the edge 20′ of the adjusting wheel 15′, and the graduations 18 are on the body of the housing 32.

In addition, the distal end is provided with an extension element 28′ constituted by a cylindrical ring snap-fitted onto the body 3 and having a length g such that it allows the cannula 7a of needle 7 to protrude by a length 1 equal to the desired insertion.

It is understood that, as shown schematically in FIG. 6a, by positioning the wheel 15′ from right to left in the drawing, in positions 1, 2 and 3 it is possible to carry out injections, respectively, of volume V1 in position 1, of volume V3-V1 in position 2, and of volume V6-V3 in position 3.

This injection device allows pre-set, precisely dosed injections to be given to specific areas of a patient's body, making intradermal or subcutaneous injections easy, so that they can be performed by an inexperienced user or one with dexterity problems, or by the patient himself as a self-injection.

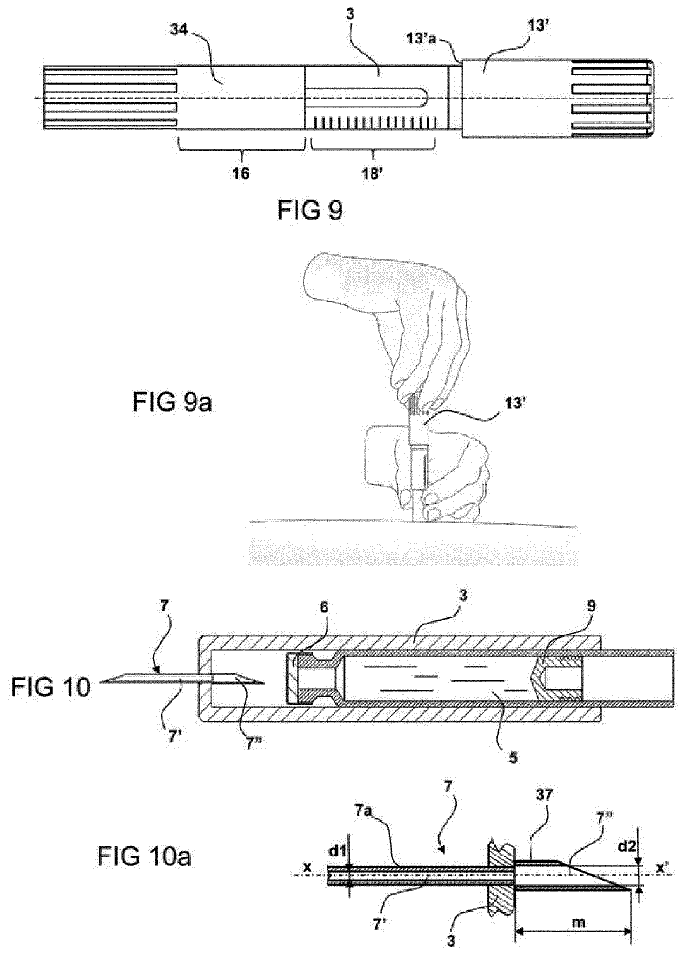

FIGS. 8 to 9 show another embodiment of an injection device according to the present invention which fundamentally has a tubular body 3, inside which there is housed a reservoir 5 in which a plunger 9, of the type described above, is mounted so as to be longitudinally movable under the action of a plunger rod 11a constituted by a screw. At its distal end, this plunger rod 11a is fixed to a plunger 9 and extends through a nut 14 integral with the body 3 to terminate at its proximal end in a bell-shaped head 13′. The latter covers the plunger rod 11a and part of the body 3.

Under these conditions, it is understood that the rotation of the head 13′ controls the longitudinal displacement of the plunger 9 in the reservoir 5 and the expulsion of a given volume of treatment product through the injection needle 7.

This injected volume is controlled by means of graduations 18′ arranged on the body 3, and the index can be formed here by the distal edge 13′a of the head 13′ as shown in FIG. 9. This embodiment is especially beneficial in that it allows reading on the posterior or proximal part of the body 3, the anterior or distal part of the body 3 being assigned to the whole-handed gripping in an area 16, hereinafter referred to as the “distal holding area”.

Of course, according to the invention, any other measuring system may be used as long as it is located outside the area in which the user holds the body 3 in his whole hand, i.e. in the distal holding area 16. Such a measuring system can also be constituted by longitudinal indexes arranged respectively on the body 3 and on the head 13′, which may coincide with each given turn with the screw 11′.

In this exemplary embodiment, and as shown in FIGS. 8, 8a and 8b, the injection needle 7 comprises a base 36 to which its cannula 7a is attached, for example by gluing, press-fitting, welding, for example electron beam welding. This base 36 is formed on the one hand by a collar 36a having the shape and dimensions of the septum which usually closes the reservoirs, especially constituted by standard cartridges. The proximal face of this collar extends inward from the neck 5a of the cartridge through a boss 36b. As shown in FIGS. 8, 8a, and 8b, this boss has a truncated cone-shaped cavity 36c that converges from the proximal side to the distal side.

The boss 36b is of such a diameter that it fits into the neck 5a of the cartridge and the 36a base is attached to the neck 5a of the cartridge by crimping means such as a ring 39, as is the septum on conventional cartridges.

It can be seen that such an embodiment provides two functions that are not found in the previous prior art.

Firstly, it makes it possible to reduce the unnecessary length of the 7a cannula as much as possible by allowing the proximal end of the latter to be almost flush with the outlet neck 5a of cartridge 5.

Secondly, the convergent design formed by the cavity 36c favours the flow of the product to be injected.

Thus, these two new functions generated by such an embodiment cooperate to reduce the importance of the pressure drop imposed on the injected product during the injection operation, which makes it possible, for a given internal diameter of the cannula 7a, to reduce the force necessary to inject the product, or for a given injection force to reduce the internal diameter of cannula 7a.

Of course, depending on the invention, the same functions could be implemented with reservoirs other than standard cartridges, such as syringes or other conventional containers made of glass, plastic or any other material, moreover whatever their respective volumes.

This device makes it possible to inject products that are much more viscous or even pasty than many commercial devices, while using conventional containers. Injecting such viscous or semi-solid products makes it possible to reduce the volume of administration for certain patients requiring high therapeutic doses, in oncology for example, with monoclonal antibodies. This high viscosity makes it possible to increase the concentration of active ingredient and, for example, to go beyond their solubility to subcutaneous solutions in which the molecules of these active ingredients could have specific arrangements, contributing to their concentration, stability and injectability, for example by reducing the shear stresses applied during their administration. This device thus facilitates especial injections, such as intra-articular or intravitreal injections, or even the injection of biological glues or fillers for wrinkles, with greater strength and precision with finer needles, even for products that are more viscous with a longer duration of action.

The device according to the invention makes it possible to control the speed of administration according to the patient's feeling (pain), and to make pauses during administration. For an injection, due to the high stability at the injection site, it is possible to wait a little before continuing the administration without making parasitic movements of the inserted needle, which at the moment is difficult with current injection devices operating in translation.

In the operating mode shown in FIG. 8 the body 3 is provided with a cap 34 which fits over it and which allows the needle 7 to be covered. This cap has an internal insert 35, especially made of rubber or butyl, which, in the closed position, rests on the bevel of the cannula 7a of the needle 7 so that the cartridge 5 is sealed.

Of course, the cap 34 might not comprise any systems that come into contact with the cannula 7a of the needle 7, for example if the product to be injected is of a viscosity that does not allow it to flow through the cannula without pressure. This makes it possible to obtain a needle pre-assembled on the reservoir which has not experienced any contact prior to insertion.

This cap, due to its ergonomic design, will allow the device to be capped according to the invention with only one hand and left lying on a table, thus avoiding the risk of needle-prick injury. Also, it cannot be activated by a child.

In another embodiment of the invention, the reservoir may be a standard pre-filled syringe, for example of the Hypak type of 0.5 mL, with a diameter smaller than that of standard cartridges, comprising a distal Luer end closed by a standard butyl stopper, and holding fins at its proximal end.

Although they have a smaller internal diameter than standard cartridges, more suitable for the administration of viscous products, 0.5 mL syringes have the disadvantage of not being Luer-locked at their distal end, or of not having any specific way of being held when injecting viscous products.

Thanks to the device according to the invention, it is possible to obtain a conventional but more resistant Luer-lock assembly, without any risk of rotation or disconnection of the needle, this design therefore being more suited to viscous products.

The syringe is inserted into the body 3, which comes into contact with the skin during the injection. In this specific embodiment, the distal part of the body 3 is terminated by a male Luer-lock-type tip, without Luer cone; the Luer cone of the 0.5 mL syringe as well as its specific butyl stopper protrude through this distal end. The glass wings of the proximal end of the syringe can circulate in two notches formed on either side of the proximal part of the body 3, up to a certain distance 1 where the wings come to a distal stop in notches in a low position before use. At this point, the Luer cone of the needle is exposed through the distal part of the body 3, and the butyl stopper can be removed and replaced with a standard needle. Attachment of the rotating needle in the Luer lock causes the syringe to move in translation within the body 3 through the notches to a high locking position, in which the wings come into proximal abutment. The movement of the wings within the body 3 serves as a visual check to ensure that the needle is assembled. In this new arrangement, the fragile fins of the glass syringe can no longer withstand the full pressure force during the injection, which is distributed over the distal part of the glass syringe from the outer surface of the Luer cone through the plastic base and the Luer-lock part of the plastic body in the distal area to the proximal end of the body 3. This new, stronger assembly around the Luer-lock end allows higher injection forces to be exerted. It is also possible to use this new Luer-lock assembly in a conventional syringe version, with a body terminated at the proximal end by a standard finger rest and plunger rod.

In another embodiment of a device according to the invention, around a 0.5 mL syringe, this device may have the same functions as those described above, but in this especial case, the device will preferably be connected to the pre-filled syringe by lateral clipping; this makes it possible to add a specific part which can be clipped onto the cone of the base of the needle to control and ensure its correct insertion and retention on the Luer end of the syringe. This clipped part of the body 3 may comprise the other elements according to the invention with the functions already described, such as the resting on the skin at its distal end or the possible presence of an adhesive protection system.

This specific part of the device that clips laterally on the base ensures a sufficient connection between the syringe and the needle to allow the injection of viscous products. In some applications, the device according to the invention may also be extended at its proximal end by a finger rest associated with a plunger rod to allow its use as a conventional syringe.

Still around a 0.5 mL pre-filled syringe reservoir, it is possible to provide a fixed needle version, comparable to that envisaged for cartridges. In this especial case, a needle with a metallic base can be inserted through the proximal opening of the reservoir. This base has a shape suited to the internal diameter of the reservoir so as to reach a position of abutment inside the syringe on the Luer-end side. The cannula of this needle then protrudes through the opening of this end; it is possible to plug this cannula with a suitable butyl stopper protecting the product to be administered until injection. The main advantage of this arrangement is to offer a version with a needle of the desired diameter already installed, without any contact with any plastic.

Likewise, compared to the previously described version with the standard needle attached by its plastic cone, this solution can also reduce the dead volumes as well as the injection forces required to administer the same product. It may also be possible to combine it with a vein testing device by providing an opening or air escape point between the cannula and the product to be injected that is too small in diameter for viscous product to flow through, but large enough for air to pass through and cause blood to rise.

This fixed-needle solution can be adapted both to the screw system described above in accordance with the invention and to a standard syringe-type arrangement with a conventional plunger rod.

FIG. 10 shows an injection device of which the body 3 receives a standard cartridge 5 of which the septum 6 is intended to be pierced by a needle 7 when the cartridge 5 is inserted into the body 3. In this example, and as shown in FIG. 10a, the needle 7 is constituted of two parts, namely an outer and anterior part 7a of conventional type and of an internal diameter d1 depending on the product to be injected and the operation to be performed, and an inner and posterior part 37 of larger internal diameter d2. The two parts, anterior 7a and posterior 37, of the needle 7 are joined together, for example by gluing or welding. As shown in FIGS. 10 and 10a, the anterior part 7a of the needle passes through the distal wall of the body 3 in such a way that its posterior part 37 of larger diameter comes into contact with the inner face of said distal wall 3. The free end of the posterior part 37 ends with a bevel so as to improve its capacity to perforate the septum 6 when it is placed in the body 3.

The length m of the posterior part 37 of larger diameter shall be kept as short as possible, i.e. slightly greater than the thickness of the septum of the cartridge that the body 3 is intended to receive, so as to allow it to just pass through the septum when the cartridge is placed in position.

It is therefore understood that the larger-diameter posterior part 37 performs several functions.

Firstly, it provides better retention of the needle 7 in relation to the distal part of the body 3, since this internal part 37 forms a kind of shoulder.

Secondly, as the diameter d2 of its internal duct is greater than the diameter d1 of the duct of the anterior part 7a, the pressure drop suffered by the injected product during the injection will be lower than it is according to the prior art when the posterior part 37 has the same diameter as the external part.

Finally, by giving this posterior part 37 such a length that it can just pass through the septum 6, the pressure drop suffered by the product during the injection is further reduced, and the injection force required is therefore reduced. As explained above, this makes it possible either to reduce the internal diameter of the needle or to increase its length, or to inject products that are less fluid through a needle diameter smaller than those currently used.

Although the injection device according to the invention has been described above in relation to examples in which the reservoirs containing the product to be injected were constituted by cartridges, it can of course also be used with reservoirs constituted by standard or non-standard syringes.

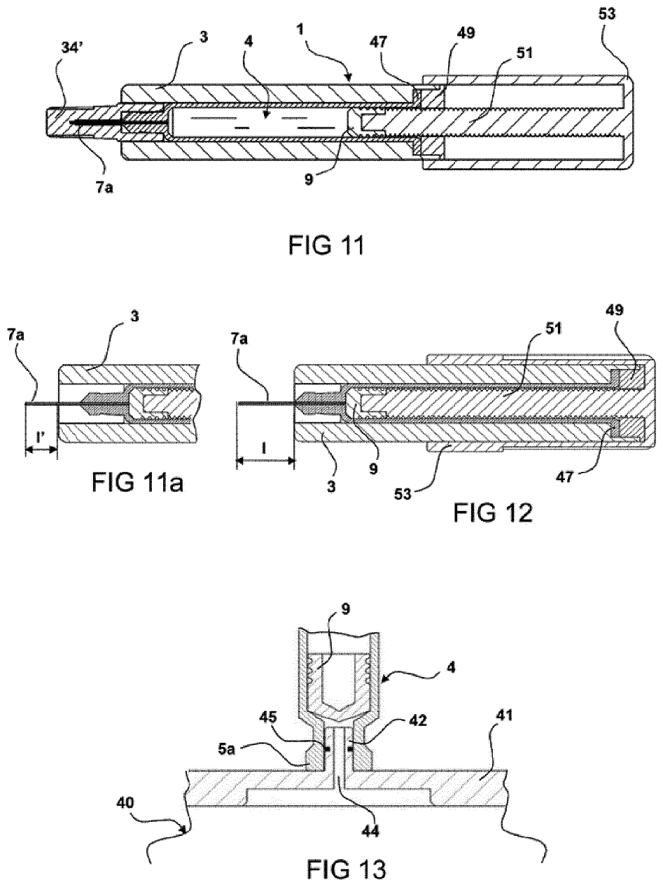

FIGS. 11, 11a and 12 show such an injection device 1 comprising a tubular body 3 into which there is inserted a standard syringe 4 ending in a fixed needle of which the cannula 7a is protected by a tight cap 34′, especially made of butyl. The proximal end 47 of this syringe is held in the housing 3 by a nut 49 which is itself fixed to the proximal end of the body 3. The nut 49 is penetrated by a thread in which a threaded rod 51 is screwed, the distal end of which is attached to the plunger 9 of the syringe 4.

The other end of the threaded rod 51 is an integral part of the bottom of a bell-shaped cap 53 of which the dimensions are such that, in the position removed from the plunger (FIG. 11), it slightly covers the body 3. Under these conditions the injection is carried out by simply rotating the cap 53 in relation to body 3.

The length of the body should be such that its distal end reaches the insertable end of the needle cannula 7a and exposes a given length 1 of the needle, as shown in FIGS. 11 and 12.

Furthermore, according to the invention, it is understood that by adjusting the length of the body 3 it is possible, for a given syringe equipped with a needle whose cannula 7a has a given length, to adjust the length 1′ of the insertable part thereof, as shown in FIG. 11a.

The present injection device thus makes it possible, on the one hand, to control the insertable length of the cannula 7a and, on the other hand, to ensure stable positioning on the patient's skin during the injection phase, especially due to the bearing surface of the body 3 on it and the grip achieved by the user.

The present invention also relates to a method and a filling device for an injection system.

In a first embodiment, means have been shown in FIG. 13 to ensure the filling and packaging of an injection system 1 or its reservoir 5 from the neck 5a of the latter, i.e. the opposite of the filling direction usually used according to the prior art. Such a filling process is especially beneficial when the product whose reservoir 5 is to be filled is of a high viscosity.

This filling device comprises a storage container 40 which is provided with a filling head 41 which comprises a connecting nozzle 42 of a diameter enabling it to be inserted into the neck 5a and which is pierced by a supply duct 44. The tight fit between the nozzle 42 and the neck 5a of the injection device can be ensured by a seal, for example of the O-ring type 45, which is fitted in a circular groove of said nozzle 42.

Before filling, as shown in FIG. 13, the plunger 9 of the injection device 1 is in contact with the inlet of the neck 5a. If the treatment product is injected through the feed line 44 during the filling phase, the plunger 9 is pushed, so that the desired product dose is filled without the disadvantage of an air space being trapped between the plunger and the treatment product.

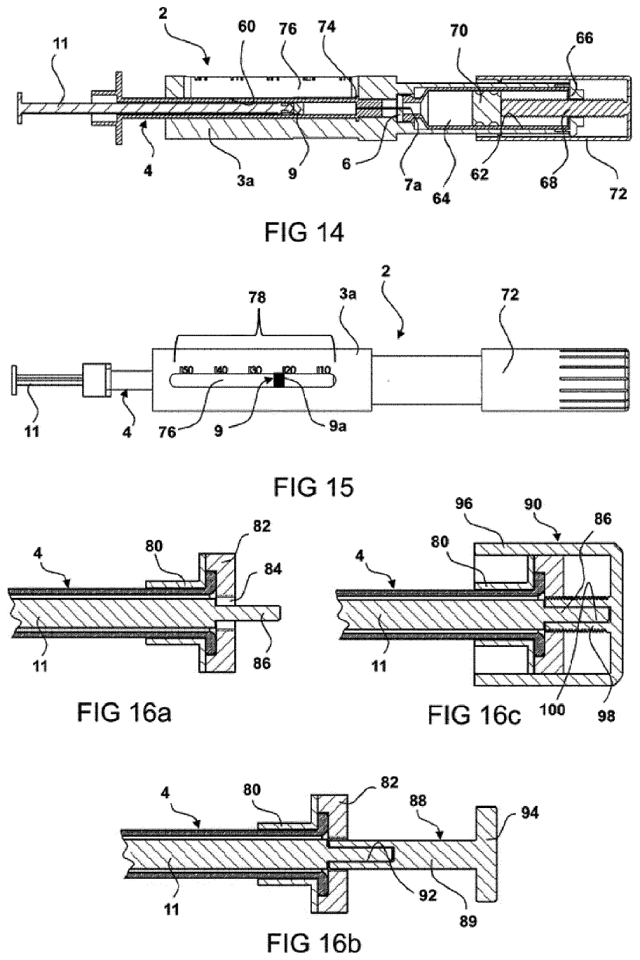

In a second embodiment, FIGS. 14 and 15 show a filling device 2 of a standard-type syringe 4 which allows the syringe to be filled from its distal end, i.e. through the cannula 7a of its needle.

This filling device is constituted by a cylindrical receiving housing 3a which is pierced at each of its ends by a longitudinal cylindrical receptacle, namely a receptacle 60 formed in the proximal part of the receiving housing and which is able to receive the syringe 4, and a receptacle 62 formed in the distal part and able to receive a filling reservoir 64 which is closed in the conventional manner by a septum 6 of the same type as that shown in FIG. 10.

This filling reservoir 64 is held in position in the receptacle 62 by a nut 66 which is screwed into the housing 3a and which is penetrated by a thread into which a threaded rod 68 is screwed, the distal end of which is connected to a plunger 70.

The other end of the threaded rod 68 is an integral part of the bottom of a bell-shaped cap 72 of which the dimensions are such that when removed from the plunger 70 it slightly covers the housing 3a.

The distal part of housing 60 receiving syringe 4 terminates in a boss 74 which forms a stop against which the syringe comes into contact when it is inserted into said receptacle 60. This boss 74 is arranged in such a way that, when the syringe 4 is in contact with it, its cannula 7a has perforated the septum 6 of the filling reservoir 64 and has penetrated the latter.

Under these conditions, the syringe is filled with a determined dose of treatment product in the following way. First, the plunger rod 11 of the syringe 4 is pushed so that its plunger 9 abuts against the bottom of the syringe, then the cap 72 is rotated, which has the effect of pushing the plunger 70 and expelling a given quantity of treatment product into the syringe 4 through its cannula 7a, pushing the plunger 9 of the syringe 4.

It is understood that this method of filling has the first advantage of avoiding the creation of an air pocket between the plunger 9 of the syringe and the treatment product.

In addition, the quantity of treatment product injected into syringe 4 can be monitored with the aid of means for controlling the longitudinal displacement of the cap 72 relative to the housing 3a, which may be means of the type used in the device as shown in FIG. 9.

As shown in FIG. 15, it is preferable to make a longitudinal window 76 through the housing 3a so that the syringe 4 and especially the position of its plunger 9 can be seen through said window. On the housing 3a, and at the edge of the window 76, graduations 78 should also be made, spaced in such a way that they correspond to the doses of product to be injected. The user of the syringe will thus screw on the cap 72 until the distal part 9a of plunger 9 of the syringe is opposite the graduation 78 corresponding to the quantity of treatment product he wishes to inject, i.e. for example 20 units in FIG. 15.

Once the desired dose has been loaded into the syringe, the user will remove the syringe from the filling device 2 and can perform the injection. The user can easily start again with another syringe by inserting the syringe into the receptacle 60 of the filling device 2 and proceeding as described above.

This method of filling a syringe, especially a standard syringe, and the filling device used to carry out this method are especially beneficial when repeated injections or variable daily quantities of treatment products, such as insulin or growth hormones, have to be implemented due to the ease of use, making them suitable for use by the patient himself/herself.

Furthermore, it has been verified in tests that this arrangement gives an accuracy comparable to that provided by conventional pen-injectors and furthermore facilitates the injection of large volumes of treatment product without being subject to the dose limits and injection time constraints of said pen-injectors. This allows a manual injection to be carried out from an injection device filled by means controlled by rotation which can perform all the functions of conventional pen-injectors using simple standard syringes and which can be even smaller, simpler, lighter, robust and therefore less expensive.

It is known that for some injectable products it may be necessary to mix two base products prior to the injection, especially in the case of extemporaneous preparation, such as a reconstitution for certain compound formulations. It may also be necessary to transfer all or part of a treatment product from a primary packaging to a reservoir which will then be used to carry out the injection.

The most common method is to use two syringes, each containing one of the two products to be mixed, which are joined together by means of a connector. The products are then moved back and forth from one syringe to the other, resulting in a homogeneous mixture. All of the mixed product is then transferred to one of the two syringes, which is used as an injection syringe.

It is understood that while multiple transfers of base products from one syringe to another can be easily and quickly accomplished when the plunger rods of these syringes are pressure-actuated, this is not the case when the plunger rods are actuated by rotation, as is the case for example in the devices described by way of example in FIGS. 8, 9, 11 and 14.

The present invention, as shown in FIGS. 16a, 16b and 16c, makes it possible to overcome this disadvantage and makes it possible to easily ensure mixing before injection of two base products while allowing injection by a rotary action on its plunger rod.

This injection device consists in this case of a syringe 4 of which the proximal end is held between a circular flange 80 and a holding plate 82 fixed to it by any means (not shown in the drawing), and which is penetrated by a threaded hole 84, the diameter of which is slightly larger than that of the plunger rod 11 of this syringe. The proximal end of this plunger rod 11 ends in a cylindrical pin 86 of a diameter smaller than that of the plunger rod 11. The injection device thus comprises, in addition to the syringe 4, two control heads, namely a pressure control head 88 and a rotary control head 90.

The pressure control head 88 constituted by a cylindrical rod 89 with a diameter smaller than the threaded hole 84 and close to that of the plunger rod 11, this cylindrical rod being hollowed out at its distal end by an axial recess 92 capable of allowing it to receive the pin 86 and the proximal end of which is formed by a flattening 94, as shown in FIG. 16b. It is understood that the injection device thus constituted is capable of functioning as a pressure-controlled syringe, which makes it capable of easily and rapidly carrying out the aforementioned syringe-to-syringe transfers.

The rotary control head 90 has, as in the previous embodiments shown in the above-mentioned drawings, a bell-shaped cap 96, the bottom of which has an axial screw portion 98 suitable for screwing into the threaded hole 84 and which is penetrated by an axial housing 100 suitable for receiving the pin 86, as shown in FIG. 16c. It is understood that, when formed in this way, the injection device is capable of functioning as a rotation-controlled syringe, since a rotation in the direction of the screwing of the cap 96 has the effect of pushing the plunger rod 11, thus causing the injection of the treatment product contained in the syringe 4.

The present embodiment is beneficial in that, by simply exchanging the control head, the user can easily and quickly switch products back and forth from syringe to syringe for mixing and injection of treatment product by means of a rotationally controlled injection device.

It is known that when conventional injection devices, such as syringes or cartridges, whether single or double-chamber, are used to perform intramuscular injections, it is necessary to ensure that the administration is not performed in a vein. This is why, once the needle has been inserted, the practitioner performs a check, known as a “vein test”, by aspirating with the plunger of the syringe. In the event of a blood return, which means that blood is flowing back into the syringe, the practitioner confirms that the needle is in a vein and that, consequently, he or she cannot perform the injection.

In injection devices in which the injection is not by pressure but by rotation, the aspiration effected by the withdrawal of the plunger is more difficult to achieve, especially in cases where the injection products are viscous or highly viscous.

However, the applicant has found that in the case of a glass cartridge for example, when the crimping ring is sealed against the neck of the reservoir, the contact between these two components, because of their metal-on-glass composition, although it prevents the passage of liquid or semi-solids, is not completely airtight. Under these conditions, if the user inserts the needle into a blood vessel, the blood pressure causes the blood to rise in the needle, with air escaping through the reservoir-crimp ring junction. The blood then takes the place of the air in the reservoir, staining its distal part visible to the practitioner. As with a standard injection device of the syringe type, the presence of blood in the reservoir indicates that it is not possible to perform the injection at that specific location.

Under these conditions, it is understood that the rotational injection modes according to the invention allow the vein test to be carried out automatically.

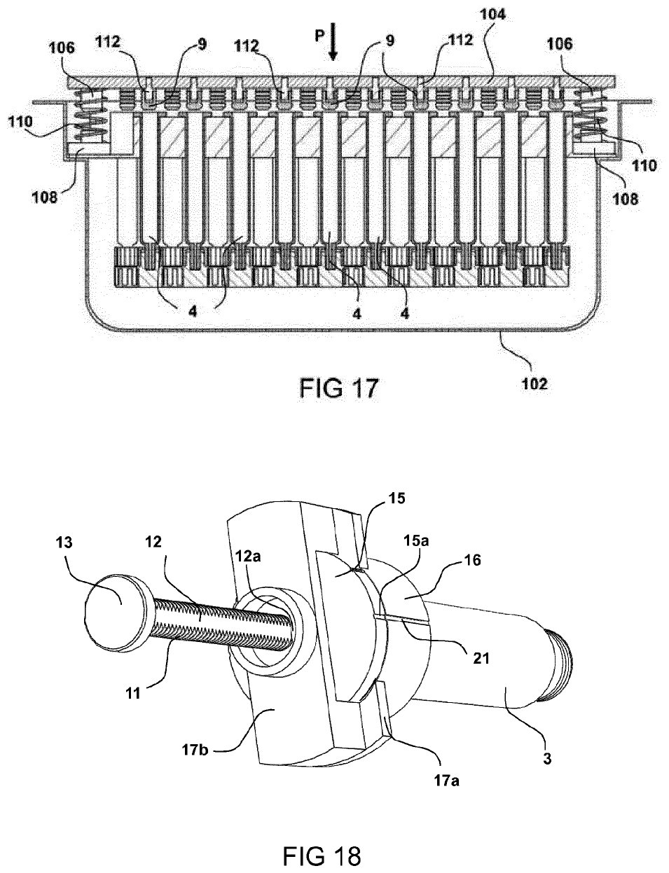

A device for packaging syringes or cartridges commonly referred to as a “nest” is shown in FIG. 17 and is typically used for the filling of products in liquid form or products of low viscosity.

According to the invention, this device is used to ensure the packaging of a series of syringes or cartridges intended to contain, especially, a first base product intended to be subsequently mixed with a second base product in order to carry out an extemporaneous preparation of an injectable treatment product, the first base product being able to be presented especially in dry or freeze-dried form.

The packaging device thus comprises the “nest” which, in a known manner, comprises a tub 102 closed by a lid 104 and containing means capable of holding vertically a set of reservoirs, consisting of cartridges or syringes 4, which it is desired to fill and package. The lid 104 is provided with means for centering in position, which means are constituted by four male studs 106 fixed in the corners of the lid and beneath its lower face and which are capable of being received in four female studs 108 arranged on the tub 102 opposite the male studs. Helical compression springs 110 surround the male and female studs and ensure, when they are in the uncompressed position (FIG. 17), that the lid 104 is held in the open position.

According to the invention, the lower surface of the lid 104 is provided, in line with each of the syringes 4, with a protuberance 112 perpendicular to the surface of the lid, which protuberance is intended to frictionally receive a plunger intended to subsequently constitute the plunger 9 of the syringe 4.

The packaging process according to the invention is carried out as described below. First of all, the syringes are filled with any conventional known device, then any drying or freeze-drying operations are carried out, with the springs 110 holding the lid 104 in position during this operation. At the end of the drying or freeze-drying operation, pressure P is exerted on the outer surface of the lid 104, against the force exerted by the springs 110, so that each plunger 9 is inserted into the reservoir of the syringe 4 with which it is opposite. This operation may be carried out inside a chamber or freeze-dryer kept under a calculated vacuum of air or inert gas. The vacuum can then be broken to return to ambient pressure, so that the latter exerts a force on each plunger 9, which has the effect of holding the plunger in each reservoir when the pressure P applied to the lid 104 is stopped and each spring 110 acts to raise it. The product is then packaged in syringes 4 and can be mixed with a liquid fraction and then injected using preferably a device according to the invention as described above in FIGS. 16a, 16b and 16c.

The present invention also has relates to a passive safety injection device intended to allow increased stability of its positioning during injection and protection of its injection needle before, during, and after injection.

Such a safety device is said to be passive because, on the one hand, its operation is entirely independent of the way in which the user carries out the injection and, on the other hand, the user cannot carry out operations or errors that would prevent its activation, such as not completing an injection before withdrawing the needle.

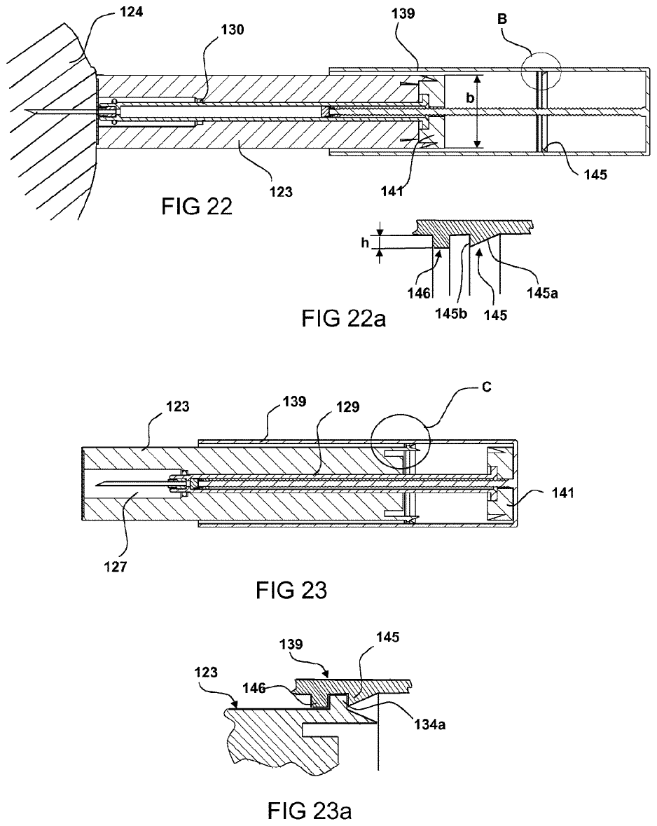

A specific embodiment of such an injection device is shown in FIGS. 20 to 23a.

This injection device 121 comprises a tubular body 123 which is hollowed out by an axial cylindrical duct 125 which opens into a cylindrical distal chamber 127 of larger diameter. The duct 125 receives a reservoir, constituted in this embodiment by a syringe 129 containing the product to be injected which ends in an injection needle 131. The distal end of the syringe 129 is provided with an O-ring seal 132 which is embedded in a seat 130 in the body 123, and the length a of chamber 127 is such that in the rest position, or pre-injection position, as shown in FIG. 1, the injection needle 131 is housed inside chamber 127.

The proximal end 123b of the body 123 ends in two diametrically opposed hooking tabs 134 which themselves end in an external boss 134a of height H followed by a bevel 134b, these tabs being made deformable by the presence of an internal space 135.

A plunger 136 is slidingly mounted inside the syringe 129 by means of a threaded plunger rod 137. The proximal end of this plunger rod is integral with the bottom of an actuating member consisting of a cylindrical cap 139, the internal diameter D of which is slightly larger than that d of the body 123 plus the height of the two stops 134a, so as to allow the cap 139 to slide on the body. Thus, D=d+2×H. In the rest position (see FIG. 1) the distal end of this cap 139 is slightly engaged on the body 123.

On the inside of the cap 139 there is a first circular distal stop 146 of height h, of rectangular cross-section, to keep the body 123 locked in the cap 139 when the needle is removed from the body 124 after administration of the product is complete. Finally, the inner face of the cap has a second circular proximal stop 145 of height h, the proximal face 145a of which is preferably inclined and the distal face 145b of which is radial, and spaced apart from the first stop 145 by a distance equal to the width of the boss 134a, and which is intended, as explained below, to prevent a second exit of the needle 131 once the injection process has been completed.

A cylindrical nut 141, of thickness e, forming a stop is screwed onto the plunger rod 137 and comprise a seat 140 in which the proximal end of syringe 129 is fixed. The periphery of the distal face of the nut 141 is hollowed out by a circular groove 143 of complementary shape to that of the bevel 134b of the deformable tabs 134. The diameter b of this nut 141 is slightly smaller than the inside diameter D of the cap 139 from which the heights H of the two bosses 134a have been subtracted. This thus gives b=D−2×H.

The distance c that exists, when the injection device is at rest, as shown in FIG. 1, between the proximal end 123b of the body 123 and the distal face of nut 141 makes it possible to control the distance by which the injection needle will emerge from the distal face 123a of the body 123 during injection, as explained below.

This distal face 123a of the cylindrical body 123 may be formed by the annular surface of the body 123 or, as in the embodiment shown in FIG. 2, may comprise an extension part 123c making it possible to increase the size of this surface.

The latter is coated with an adhesive capable of adhering to the skin 124 of the patient to be treated. For example, sticky surfaces based on silicone, acrylate, polyacrylate, or other known copolymers used in the composition of surface adhesives for medical devices, such as dressings, ostomy bags, electro-surgical electrodes and plates, patches, or capillary prostheses can be employed. This adhesive side shall be protected by a cover or a film 147.

Under these conditions the operation of the injection device according to the invention is as described hereinafter.

After removing the protective film 147, the user brings the adhesive distal end of the injection device into contact with the area of the patient's body where he or she wishes to perform the injection. This end of the injection device is then stabilised by its adhesion to the injection site.

The user then exerts linear pressure on the cap 139 in the direction of the arrow F until the nut 141 comes into contact with the proximal face 123b of the body 123, which firstly causes the injection needle 131 to be pulled out from the injection device and introduced into the body of the patient 124, as shown in FIG. 3. On the other hand, at the end of this movement in translation, the bevels 134b of the tabs 134 penetrate into the groove 143 of the nut, as a result of which the tabs 134 are deformed inwards and a rotational connection of the nut 141 to the body 123 is established. In addition, due to the inward deformation of the tabs, they are free and are not able to block the movement of the cap when, during injection, they reach the circular stops 145 and 146.

The injection device is now ready to inject the treatment product.

To do this, the user rotates the cap 139, which can be done with one hand because of the connection between the distal part 123a of the body 123 and the body of the patient 124 which is provided by the adhesive product. When the cap 139 is rotated, the valve stem 137 screws into the nut 141, and when the nut 141 is prevented from rotating relative to the body 123, the plunger 136 moves into the syringe 129 and delivers the treatment product into the patient's body until the plunger reaches the neck of the syringe 129, or the user stops the injection.

Once the injection is complete, the user pulls the cap 139 in the opposite direction to the arrow F and, with the distal face 123a adhering to the patient's skin, this pulls the syringe 129 and the nut 141 back, so that the tabs 134 are released and return to their initial position. The retraction movement is therefore carried out until the proximal face of the boss 134a of the tabs 134 comes into contact with the stop 145, and falls into the free space between the two stops 145 and 146 as shown in FIGS. 4 and 4a.

This means that any relative movement of the cap 139 in relation to the body 123 in the direction of the arrow F or vice versa is no longer possible and the injection device is thus locked and secured against further use.

By continuing the withdrawal movement, the user ensures that the distal side of the injection device is detached from the surface of the patient's skin thanks to the locking of the body 123 with respect to the cap 139 against the second distal stop 146.

It should be noted that the safety features equipping this injection device have the advantage of being much simpler and therefore easier and cheaper to achieve than current systems based, for example, on the triggering of spring mechanisms.

Of course, although this example has been described in relation to a small-volume syringe, such as an insulin-type syringe, it can be adapted to any other device using standard or non-standard syringes or cartridges, either in a screw injection mode as described in the present example or in traditional translational injection modes with conventional syringes using smooth plunger rods and finger rests.

In such a version with a translationally actuated smooth plunger rod, the cap 139 is replaced by two arms which are also integral with the plunger rod. These two arms can move through two windows inside the finger rest. As with the screw version, the needle is inserted by pushing these two arms in the direction of the arrow F.

In addition, the safety features implemented in the present injection device also have the advantage that the cap that usually covers the injection needle can be replaced. This eliminates the need to remove the cap, which is dangerous because it exposes the user to the sharp bevel, and also because it can damage the injection needle and, lastly, because there is a risk of external contamination of the needle prior to injection.

In a variant of the invention, the body 123 may include a window extending along the length of the syringe and enabling its contents to be checked before use.

In addition, and according to the invention, a label may connect the body 123 to the cap 139 in such a way as to ensure the relative retention of these two parts before injection. Such a label will also make it possible to verify before use the integrity of the seal of the injection device. This label may include a predetermined cutting line to facilitate its breakage during injection.

1-29. (canceled)

30. An injection device of the type comprising a cylindrical body (3) enclosing a reservoir (5) containing a product to be injected, said reservoir opening at one end on an injection needle (7) provided with a cannula (7a) and being closed at its other end by a plunger (9) mounted so as to be movable in translation in the reservoir under the control of a plunger rod (11, 51) provided with a control member (13, 13′, 53), the device comprising means for controlling the quantity of product to be delivered, wherein the control means are arranged on the body (3) in an area located outside an area where the body (16) is intended to be fully gripped by the hand.

31. An injection device according to claim 30, wherein the area intended to be fully gripped by the hand is located in the distal region (16) of the injection device.

32. An injection device according to claim 30, wherein the plunger rod (11) comprises a threaded portion (11a) on which an adjusting wheel (15) is screwed, and the body (3) comprises two end stops, namely a distal stop (17a) and a proximal stop (17b), between which the adjusting wheel (15) can be positioned.

33. An injection device according to claim 32, wherein the adjusting wheel (15) comprises, in a peripheral area, graduations (18) which are visible from the proximal end of the body (3), and the latter comprises at least one index (20) whose cooperation with the graduations (18) makes it possible to determine an angle of rotation of said adjusting wheel (15) and thus its longitudinal displacement with respect to the threaded portion (11a).

34. An injection device according to claim 33, wherein the area of the adjusting wheel (15) comprising the graduations (18) projects laterally from the body (3) in at least one area.

35. An injection device according to claim 33, wherein the proximal part of the body (3) at least partially covers the adjusting wheel (15) and comprises at least one transparent area through which at least one of the graduations (18) can be seen.

36. An injection device according to claim 32, wherein the proximal part of the body (3) forms a cylindrical housing (32), which is penetrated by a threaded portion (11a) of the plunger rod (11), the adjusting wheel (15′) being able to be moved by screwing or unscrewing on the threaded portion (11a) to pass from the proximal stop (17b) to the distal stop (17a), these stops being formed, respectively, by the two end walls of the housing (32) and the edge (20′) of the adjusting wheel (15′) forms an index, and graduations (18) are provided on the body (3) of the housing (32).

37. An injection device according to claim 30, wherein the reservoir (5) is constituted by a standard injection cartridge or syringe.

38. An injection device according to claim 30, wherein the plunger rod (11) comprises an element that acts frictionally on the inner wall of the reservoir (5).

39. An injection device according to claim 30, wherein the distal part of the body (3) comprises an extension element (28) which is traversed by the cannula (7a) of the injection needle (7), this cannula extending beyond the distal part of the extension element (28) by a length (1) equal to the desired insertable length of said cannula and the distal part of the extension element (28) comprises a tapered portion (30).

40. An injection device according to claim 30, wherein the plunger rod comprises at least one threaded portion (11a) which is engaged with a nut (14) integral with the body (3), and in that the control member (13′) is bell-shaped extending towards the distal end of the body (3) and capable of covering part of the latter and the distal part of the body (3) comprises a removable cap (34) provided with an internal cavity capable of receiving the injection needle (7).

41. An injection device according to claim 40, wherein the internal cavity of the cap (34) comprises an insert (35), capable of resting on the end of the needle (7) in order to close it and the insert consists of rubber or butyl.

42. An injection device according to claim 30, wherein the cannula of the injection needle is hollowed out by a channel, the cannula (7a) being fixed to a base (36), comprising an axially centred circular plate (36a) with an adjacent cylindrical boss (36b) of the same axis (xx′), which is hollowed out by a cavity (36c) in communication with the channel of the needle, this cavity (36c) forming a convergent duct passing from its proximal part towards its distal part, and
wherein the circular plate (36a) of the base (36) of the needle is applied against the distal face of a reservoir (5) by connecting means, and the connection between the base (36) and the distal face of the reservoir (5) is established in a non-tight manner.

43. An injection device according to claim 30, in which the reservoir (5) is closed by a septum (6), the cannula of the injection needle (7) being hollowed out by a channel, the cannula (7a) being integral with a cylindrical base (37) formed by a tubular element comprising a channel (7″) which is arranged in the extension of the channel of the cannula (7a), and the internal diameter (d2) of which is greater than that (d1) of the cannula, and
wherein the length (m) of the posterior part (37) of the needle is slightly greater than the thickness of the septum (6).

44. An injection device according to claim 30, wherein the body (3) comprises means (47, 49), possibly removable, for holding the reservoir (4), the length of the body (3) being such that it allows a length (1, 1′) of cannula (7a) equal to the length that it is desired to insert to protrude and the plunger rod (51) comprises at least one threaded portion which is engaged with a nut (49) integral with the body (3), the control member (53) being bell-shaped and extending towards the distal end of the body (3) and being capable of covering part of the distal end of the body.

45. An injection device according to claim 30 comprising a reservoir (4), the plunger (9) of which is moved by the plunger rod (11) which is of circular cross-section under the action of a control head (88, 90) and, at its distal end, an injection needle, wherein the proximal end of the plunger rod (11) comprises means capable of successively cooperating, in a removable manner, with, on the one hand, a pressure plunger control head (88) and, on the other hand, a rotary plunger control head (90) and comprising a nut (82) capable of being fixed to the proximal part of the reservoir (4), the diameter of the threaded hole of which nut is greater than that of the plunger rod (11) and is smaller than the internal diameter of the reservoir (4).

46. An injection device according to claim 45, wherein the pressure plunger control head (88) is constituted by a cylindrical element (89) of which the diameter is smaller than that of the threaded hole of the nut (82), so that it can be received in the reservoir (4), this cylindrical element (89) being capable of being fixed to the proximal end (86) of the plunger rod (11).

47. An injection device according to claim 45, wherein the rotary plunger control head (90) comprises a screw (98) able to be screwed into the threaded hole of the nut (82), so as to come to rest on the proximal part of the plunger rod (11) and be able to push it towards the distal end of the reservoir (4) when the screw (98) is screwed in and the screw (98) is fixed to the bottom of a cap (96).

48. An injection device according to claim 30, comprising an injection needle (7) provided with a cannula (7a) hollowed out by a channel, this cannula (7a) being fixed to a base (36), comprising an axially centred circular plate (36a) with an adjacent cylindrical boss (36b) of the same axis (xx′), which is hollowed out by a cavity (36c) in communication with the channel of the needle, this cavity (36c) forming a convergent duct passing from its proximal part towards its distal part.

49. An injection device according to claim 30, comprising an injection needle (7), provided with a cannula (7a), hollowed out by a channel, this cannula (7a) being integral with a cylindrical base (37) formed by a tubular element comprising a channel (7″) which is arranged in the extension of the channel of the cannula (7a), and the internal diameter (d2) of which is greater than that (d1) of the cannula and wherein the external diameter of the base (37) is greater than that of the cannula (7a).
